(19) United States
(12) Patent Application Publication
(10) Pub. No.: US 2021/0169319 A1
(43) Pub. Date: Jun. 10, 2021

(54) Nasopharyngeal Mirror Device

(71) Applicants: University of Washington, Seattle, WA (US); Seattle Children's Hospital d/b/a Seattle Children's Research Institute, Seattle, WA (US)

(72) Inventors: Sanjay Parikh, Seattle, WA (US); Ryan King, Seattle, WA (US); Nisha Pradhan, Seattle, WA (US); Ga Won Kim, Seattle, WA (US); Koustubh Muluk, Seattle, WA (US); Cheng-Ying Wu, Seattle, WA (US); Sarah Rudberg, Seattle, WA (US)

(21) Appl. No.: 17/110,014

(22) Filed: Dec. 2, 2020

Related U.S. Application Data
(60) Provisional application No. 62/943,592, filed on Dec. 4, 2019.
(60) Provisional application No. 63/032,876, filed on Jun. 1, 2020.

(51) Int. Cl. A61B 1/253; A61B 1/00

(52) U.S. Cl. CPC A61B 1/253; A61B 1/00163; A61B 1/00009

(57) ABSTRACT

In a first example, a nasopharyngeal mirror device includes a handle, a convex mirror configured to move with respect to the handle, and an anti-fogging device configured to reduce condensation on the convex mirror. In a second example, a method includes inserting a convex mirror of a nasopharyngeal mirror device into a mouth of a patient and moving the convex mirror with respect to a handle of the nasopharyngeal mirror device such that a tissue of interest of the patient is viewable within the convex mirror. The method also includes capturing an image of the tissue of interest with a camera of the nasopharyngeal mirror device while the tissue of interest is viewable within the convex mirror.

### CROSS REFERENCE TO RELATED APPLICATIONS

This application claims priority to U.S. provisional application No. 63/032,876 filed on Jun. 1, 2020, and also claims priority to U.S. provisional application No. 62/943,592 filed Dec. 4, 2019. The entire contents of both applications are hereby incorporated by reference.

### BACKGROUND

The adenoid is a lymphatic organ located behind a patient's nasal cavity in the nasopharynx. Especially in children, the adenoid often becomes inflamed and can cause recurrent problems such as breathing issues, infections, and/or earaches. As such, surgical removal of the adenoid, called an adenoidectomy, is a common surgical procedure. A surgeon performing an adenoidectomy will typically insert a nasopharyngeal mirror into the patient's mouth to view the adenoid that is otherwise obstructed from view.

### SUMMARY

In one example, a nasopharyngeal mirror device comprises: a handle; a convex mirror configured to move with respect to the handle; and an anti-fogging device configured to reduce condensation on the convex mirror.

In another example, a method comprises: inserting a convex mirror of a nasopharyngeal mirror device into a mouth of a patient; moving the convex mirror with respect to a handle of the nasopharyngeal mirror device such that a tissue of interest of the patient is viewable within the convex mirror; and capturing an image of the tissue of interest with a camera of the nasopharyngeal mirror device while the tissue of interest is viewable within the convex mirror.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/−0-5% of the recited value.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate by way of example only and, as such, that numerous variations are possible.

### BRIEF DESCRIPTION OF THE DRAWINGS

FIG. 1 is a block diagram of a nasopharyngeal mirror device, according to an example embodiment.

FIG. 2 is a block diagram of a computing device, according to an example embodiment.

FIG. 3 is a schematic diagram of a nasopharyngeal mirror device, according to an example embodiment.

FIG. 4 is a schematic diagram of a housing, according to an example embodiment.

FIG. 5 is a schematic diagram of a housing, according to an example embodiment.

FIG. 6 is a schematic diagram of a convex mirror, according to an example embodiment.

FIG. 7 is a block diagram of a method, according to an example embodiment.

### DETAILED DESCRIPTION

Conventional nasopharyngeal mirrors typically do not provide a full view of the adenoid and surrounding tissues and are not adjustable to accommodate the patient's unique anatomy. Additionally, the patient's or the surgeon's breath during the adenoidectomy can cause the nasopharyngeal mirror to fog up and impair the surgeon's view of the patient's tissues. Also, conventional nasopharyngeal mirrors are of limited use in an educational setting because a student is not able to share the surgeon's view of the patients' tissue during the adenoidectomy.

Within examples, a nasopharyngeal mirror device includes a handle, a convex mirror configured to move with respect to the handle, and an anti-fogging device configured to reduce condensation on the convex mirror. A method of using the nasopharyngeal mirror device includes inserting the convex mirror of the nasopharyngeal mirror device into a mouth of a patient and moving the convex mirror with respect to the handle of the nasopharyngeal mirror device such that a tissue of interest of the patient is viewable within the convex mirror. The method also includes capturing an image of the tissue of interest with a camera of the nasopharyngeal mirror device while the tissue of interest is viewable within the convex mirror.

Thus, the nasopharyngeal mirror device and methods for its use include various potential benefits when compared to conventional nasopharyngeal mirror devices.

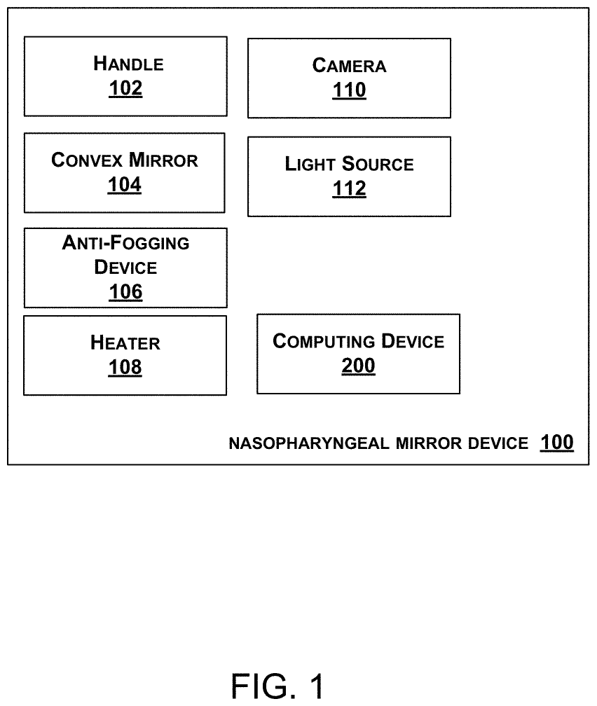

FIG. 1 is a block diagram of a nasopharyngeal mirror device 100. The nasopharyngeal mirror device 100 includes a handle 102, a convex mirror 104 configured to move with respect to the handle 102, and an anti-fogging device 106 configured to reduce condensation on the convex mirror 104. The nasopharyngeal mirror device 100 also includes a heater 108, a camera 110, a light source 112, and a computing device 200.

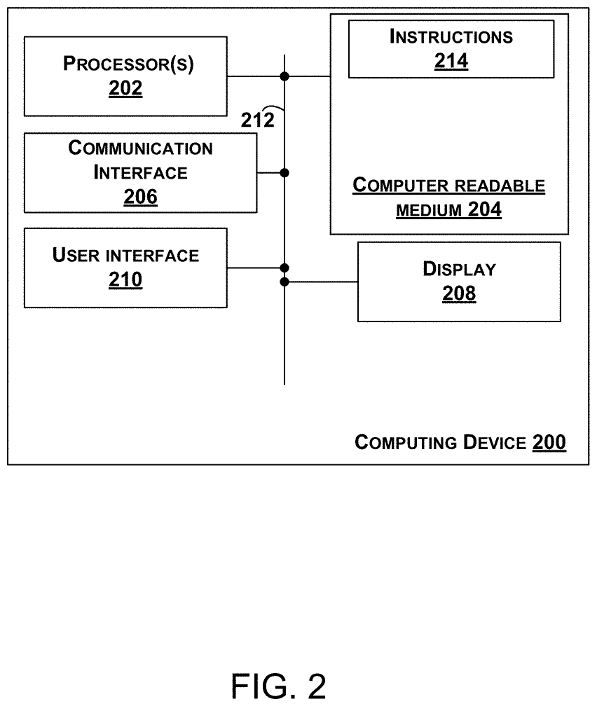

FIG. 2 is a block diagram of the computing device 200. The computing device 200 includes one or more processors 202, a non-transitory computer readable medium 204, a communication interface 206, a display 208, and a user interface 210. Components of the computing device 200 are linked together by a system bus, network, or other connection mechanism 212.

The one or more processors 202 can be any type of processor(s), such as a microprocessor, a digital signal processor, a multicore processor, etc., coupled to the non-transitory computer readable medium 204.

The non-transitory computer readable medium 204 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

Additionally, the non-transitory computer readable medium 204 can be configured to store instructions 214. The instructions 214 are executable by the one or more processors 202 to cause the computing device 200 to perform any of the functions or methods described herein.

The communication interface 206 can include hardware to enable communication within the computing device 200 and/or between the computing device 200 and one or more other devices. The hardware can include transmitters, receivers, and antennas, for example. The communication interface 206 can be configured to facilitate communication with one or more other devices, in accordance with one or more wired or wireless communication protocols. For example, the communication interface 206 can be configured to facilitate wireless data communication for the computing device 200 according to one or more wireless communication standards, such as one or more Institute of Electrical and Electronics Engineers (IEEE) 801.11 standards, ZigBee standards, Bluetooth standards, etc. As another example, the communication interface 206 can be configured to facilitate wired data communication with one or more other devices.

The display 208 can be any type of display component configured to display data. As one example, the display 208 can include a touchscreen display. As another example, the display 208 can include a flat-panel display, such as a liquid-crystal display (LCD) or a light-emitting diode (LED) display.

The user interface 210 can include one or more pieces of hardware used to provide data and control signals to the computing device 200. For instance, the user interface 210 can include a mouse or a pointing device, a keyboard or a keypad, a microphone, a touchpad, or a touchscreen, among other possible types of user input devices. Generally, the user interface 210 can enable an operator to interact with a graphical user interface (GUI) provided by the computing device 200 (e.g., displayed by the display 208).

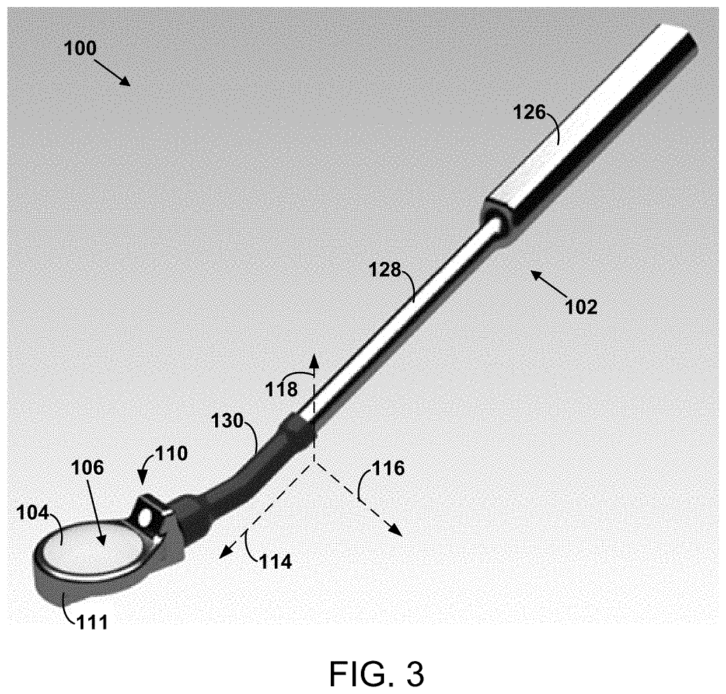

FIG. 3 is a schematic diagram of the nasopharyngeal mirror device 100. The nasopharyngeal mirror device 100 includes the handle 102, the convex mirror 104 configured to move with respect to the handle 102, and the anti-fogging device 106 configured to reduce condensation on the convex mirror 104.

The handle 102 can be formed of metal, plastic, or composite materials, for example. The handle 102 includes a first portion 126 and a second portion 128 that is between the first portion 126 and the convex mirror 104. The first portion 126 is wider with respect to a longitudinal axis 114 of the handle 102 than the second portion 128. As such, the first portion 126 provides an ergonomic gripping surface for the surgeon and the second portion 128 is more easily insertable into the patient's mouth and/or more easily maneuverable within the patient's mouth. The handle 102 also includes a third portion 130 that is bendable. The handle 102 (e.g., the third portion 130) is configured to retain a shape formed after bending.

The convex mirror 104 can take the form of a metal-coated piece of glass or plastic, for example. The metal coating faces upward in FIG. 3 and is reflective. The convex mirror 104 is mounted in a housing 111 that is connected to the second portion 128 of the handle 102 via the third portion 130 of the handle 102. Thus, the third portion 130 of the handle 102 allows the convex mirror 104 to move and/or rotate with respect to the handle 102. For example, the convex mirror 104 can rotate about one or more of the orthogonal axes 114, 116, and 118.

The convex shape of the convex mirror 104 will generally allow a surgeon to view a larger area of the patient's tissues when compared to a flat or concave mirror of similar size.

The anti-fogging device 106 can include a hydrophobic coating on the convex mirror 104. The hydrophobic coating can help reduce condensation on the convex mirror 104. The hydrophobic coating can include polytetrafluoroethylene (PTFE), fluorocarbon solids, oils, and/or any materials formed of non-polar and/or hydrophobic molecules. Additionally or alternatively, the surface of the convex mirror can be silanized to form the hydrophobic coating (e.g., treated with silane gas to coat the surface with organofunctional alkoxysilane molecules). The hydrophobic coating can be deposited concurrently with or after the reflective metal coating. The hydrophobic coating is generally transparent.

In addition, the hydrophobic coating can also be an anti-scratch coating including an oxidized aluminum (Al₂O₃) layer, for example. In some examples, aluminum can be deposited on the surface of the convex mirror 104 and heated at atmospheric pressure to form a transparent aluminum oxide (Al₂O₃) that is scratch resistant. In another example, the convex mirror 104 can include a stand-alone anti-scratch coating instead of the hydrophobic coating.

The anti-fogging device 106 can also include the heater 108 which is not shown in FIG. 3, but can be housed within the housing 111 under the convex mirror 104. The heater 108 can include a halogen bulb or a resistive heating coil, for example. The heater 108 is configured to heat the convex mirror 104 to a temperature greater than 33° C., or more specifically greater than 36° C., which can reduce condensation on the convex mirror 104.

The anti-fogging device 106 helps provide a clear view of the patient's tissues to the surgeon by reducing condensation on the convex mirror 104.

The camera 110 includes an image sensor configured to capture still images or video of tissues that are viewable upon the convex mirror by the surgeon during use of the nasopharyngeal mirror device 100. The images or video can be provided to an external monitor by the communication interface 206, for example. Thus, a student can view the actions of the surgeon in real time.

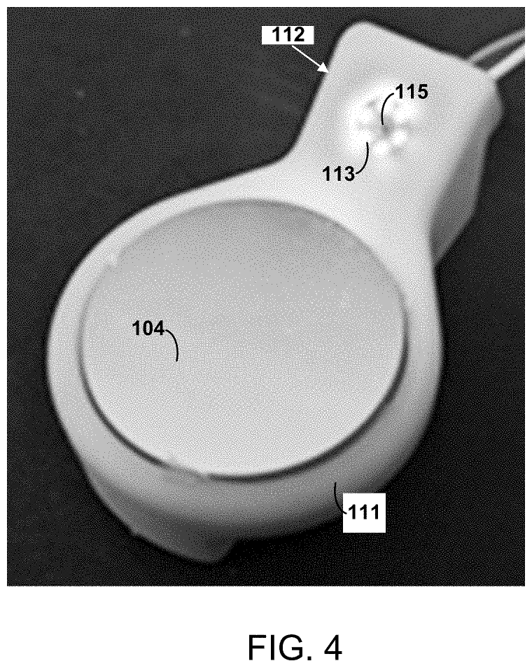

FIG. 4 is a perspective view of the housing 111. As shown, the nasopharyngeal mirror device 100 also includes the light source 112 that is configured to illuminate a field of view of the camera 110. More specifically, the light source 112 includes multiple lighting elements 113 (e.g., light emitting diodes) that encircle an aperture 115 of the camera 110.

The user interface 210 can receive a particular input that causes the light source 112 to be enabled (e.g., turned on). Additionally or alternatively, the user interface 210 can receive another input that causes the camera 110 to change its zoom setting and/or to enable (e.g., turn on) the camera 110.

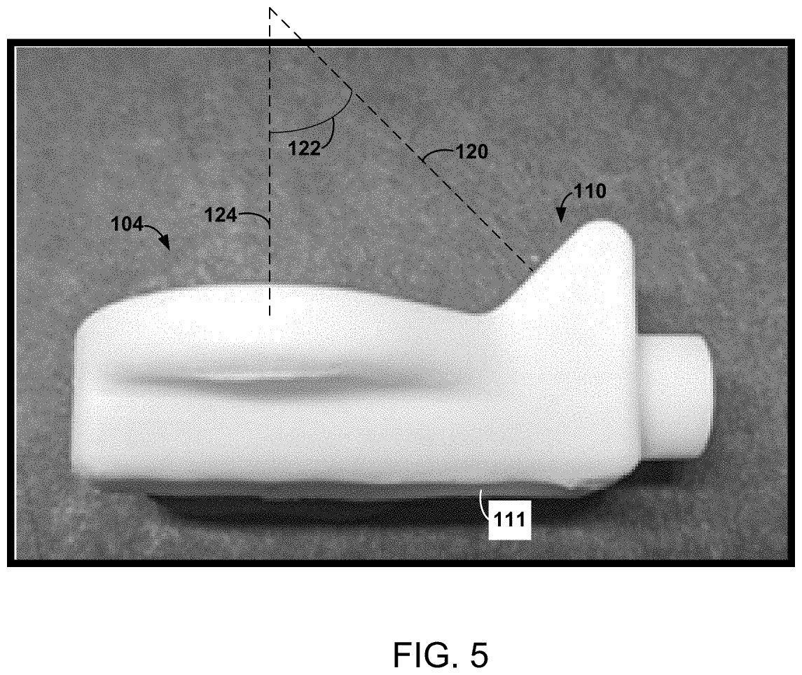

FIG. 5 is a side view of the housing 111. As shown, a first optical axis 120 of the camera 110 forms an angle 122 with a second optical axis 124 of the convex mirror 104. The first optical axis 120 is the axis of symmetry of the camera 110. The second optical axis 124 passes through a center of curvature of the convex mirror 104 and is the axis of symmetry of the convex mirror 104. In various embodiments, the angle 122 is at least 30 degrees, at least 45 degrees, or at least 60 degrees.

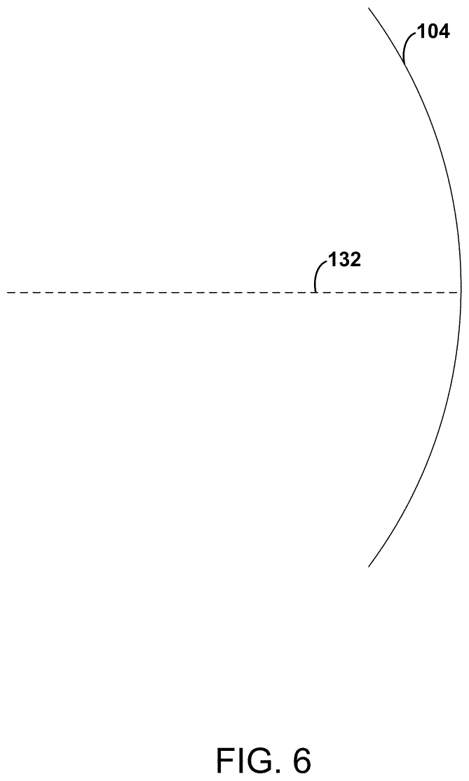

FIG. 6 is a schematic diagram of the convex mirror 104. The convex mirror 104 has a radius 132 of curvature ranging from 200-250 millimeters, or more specifically, 220-230 millimeters.

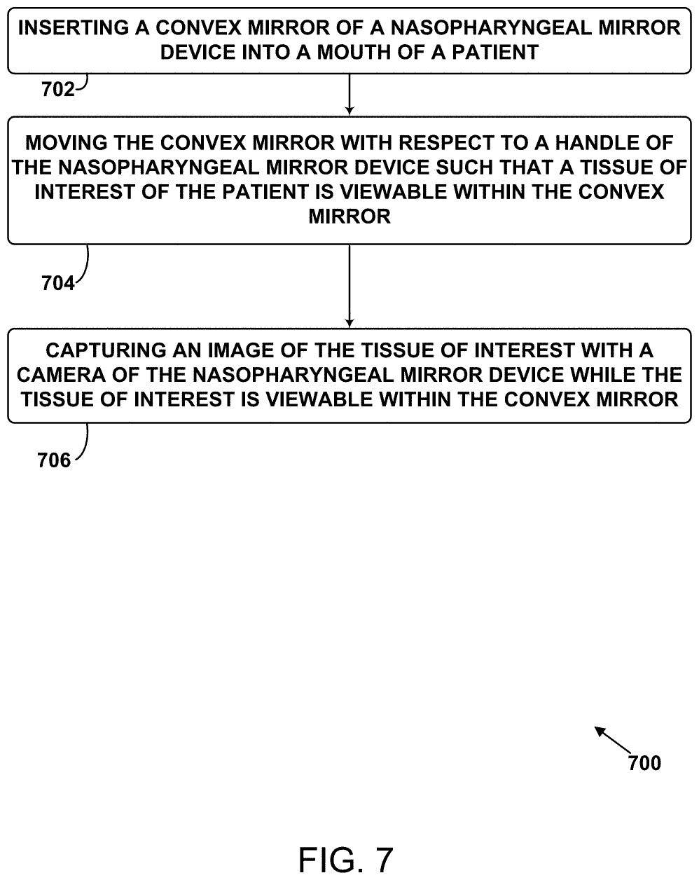

FIG. 7 is a block diagram of a method 700 of operating the nasopharyngeal mirror device 100. As shown in FIG. 7, the method 700 includes one or more operations, functions, or actions as illustrated by blocks 702, 704, and 706. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 702, the method 700 includes (e.g., a surgeon) inserting the convex mirror 104 of the nasopharyngeal mirror device 100 into a mouth of a patient.

At block 704, the method 700 includes (e.g., the surgeon) moving the convex mirror 104 with respect to the handle 102 of the nasopharyngeal mirror device 100 such that a tissue interest (e.g., an adenoid) of the patient is viewable within the convex mirror 104.

At block 706, the method 700 includes the camera 110 capturing an image of the tissue of interest with the camera 110 of the nasopharyngeal mirror device 100 while the tissue of interest is viewable within the convex mirror 104.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

What is claimed is:

1. A nasopharyngeal mirror device comprising:
a handle;
a convex mirror configured to move with respect to the handle; and
an anti-fogging device configured to reduce condensation on the convex mirror.

2. The nasopharyngeal mirror device of claim 1, wherein the anti-fogging device comprises a hydrophobic coating on the convex mirror.

3. The nasopharyngeal mirror device of claim 2, wherein the hydrophobic coating is also an anti-scratch coating.

4. The nasopharyngeal mirror device of claim 1, wherein the anti-fogging device comprises a heater that is configured to heat the convex mirror.

5. The nasopharyngeal mirror device of claim 4, further comprising an anti-scratch coating.

6. The nasopharyngeal mirror device of claim 4, wherein the heater is configured to heat the convex mirror to a temperature greater than 33° C.

7. The nasopharyngeal mirror device of claim 4, wherein the heater is configured to heat the convex mirror to a temperature greater than 36° C.

8. The nasopharyngeal mirror device of claim 1, wherein the convex mirror is configured to move with respect to the handle about three axes that are orthogonal to each other.

9. The nasopharyngeal mirror device of claim 1, further comprising a camera.

10. The nasopharyngeal mirror device of claim 9, further comprising a light source configured to illuminate a field of view of the camera.

11. The nasopharyngeal mirror device of claim 10, wherein the light source comprises multiple lighting elements that encircle an aperture of the camera.

12. The nasopharyngeal mirror device of claim 10, further comprising a user interface configured for enabling the light source.

13. The nasopharyngeal mirror device of claim 9, wherein a first optical axis of the camera forms an angle of at least 30 degrees with a second optical axis of the convex mirror.

14. The nasopharyngeal mirror device of claim 9, wherein a first optical axis of the camera forms an angle of at least 60 degrees with a second optical axis of the convex mirror.

15. The nasopharyngeal mirror device of claim 9, further comprising a user interface configured for one or more of zooming the camera or enabling the camera.

16. The nasopharyngeal mirror device of claim 1, the handle comprising a first portion and a second portion between the first portion and the convex mirror, the first portion being wider with respect to a longitudinal axis of the handle than the second portion.

17. The nasopharyngeal mirror device of claim 1, wherein the handle comprises a portion that is bendable.

18. The nasopharyngeal mirror device of claim 1, wherein the handle is configured to retain a shape formed after bending.

19. The nasopharyngeal mirror device of claim 1, the convex mirror having a radius of curvature ranging from 200-250 millimeters.

20. A method comprising:
inserting a convex mirror of a nasopharyngeal mirror device into a mouth of a patient;
moving the convex mirror with respect to a handle of the nasopharyngeal mirror device such that a tissue of interest of the patient is viewable within the convex mirror; and
capturing an image of the tissue of interest with a camera of the nasopharyngeal mirror device while the tissue of interest is viewable within the convex mirror.
